(19) United States
(12) Patent Application Publication
(10) Pub. No.: US 2025/0011841 A1
(43) Pub. Date: Jan. 9, 2025

(54) DETECTION OF A BIOMOLECULAR PROCESS

(71) Applicant: LUMICKS DSM HOLDING B.V., Amsterdam (NL)

(72) Inventors: Marco SIMONETTA, Amsterdam (NL); Rosalie Paula Catharina DRIESSEN, Amsterdam (NL)

(73) Assignee: LUMICKS DSM HOLDING B.V., Amsterdam (NL)

(21) Appl. No.: 18/711,548

(22) PCT Filed: Nov. 15, 2022
(86) PCT No.: PCT/EP2022/082010

§ 371 Date: May 17, 2024

(30) Foreign Application Priority Data
Nov. 22, 2021 (NL) 2029858
Sep. 14, 2022 (EP) 22195699.8

(51) Int. Cl. C12Q 1/6818

(52) U.S. Cl. CPC C12Q 1/6818

(57) ABSTRACT

A method of detecting a biomolecular process is provided. The method comprises: providing a DNA strand (11) comprising one or more luminophores (3) attached to the strand (11) at one or more predetermined positions along the strand; attaching handles (7A, 7B) to the DNA strand; and trapping and/or manipulating the strand (11) by manipulating the handles (7A, 7B) in a trapping setup. The method also comprises determining a position of at least one of the one or more luminophores (3) with respect to at least part of the trapping setup and determining on the basis of the one or more luminophores (3) one or more of: a focus position of at least part of an optical detector and/or an illumination system; a focus position of at least part of an optical manipulator; a position of at least part of the strand (11); a position of a portion of the strand (11) relative to one or more other portions of the strand (11); a reaction or binding position on the strand (11) of a reagent interacting with the strand (11). The one or more luminophores (3) are covalently attached to the DNA strand. An associated DNA strand is also provided.

### TECHNICAL FIELD

The present disclosure relates to a method of detecting a biomolecular process.

### BACKGROUND

Understanding and use of biomolecular processes require knowledge of molecular genomic information, such as properties of DNA sequences and of reagents such as proteins and/or other binding moieties interacting with a DNA sequence in a DNA strand. Improvements of techniques for gaining such knowledge are therefore in high demand.

E.g. observing position-dependent interaction of a reagent with a DNA sequence may provide valuable information about the reagent and the other way around. Such information, which may comprise determining precise binding locations of molecules with respect to a genetic sequence (possibly down to the base pair level) and/or tracking motion of molecules as they move along a DNA strand can yield important insights into molecular mechanisms, specific versus non-specific/off-target binding, cooperative binding, on/off rates as function of binding location, etc.

Optical tweezers combined with fluorescence microscopy have shown to be valuable tools for such studies. See also for example Candelli et al., Phys. Chem. Chem. Phys., 13:7263-7272 (2011), “Optical tweezers—methods and protocols”, A. Gennerich (Ed.), Methods in Molecular Biology books series, Springer (2017) (DOI 10.1007/978-1-4939-6421-5), and WO 2019/151864.

However, exact localisation of a binding site along a DNA strand is cumbersome and it may even be impossible with present-day optical techniques. Moreover, it has been found that binding of a reagent to a DNA strand may occur in other locations than expected, which may be due to physical and/or environmental factors; e.g. Newton et al. found that DNA stretching induces Cas9 off-target activity (Newton et al., Nature Structural & Molecular Biology, 26:185-192 (2019)-DOI https://doi.org/10.1038/s41594-019-0188-z).

The accuracy of determining binding locations of moieties in relation the DNA sequence in such studies is currently limited by the accuracy of system calibrations, such as trap position, bead sizes and DNA tension. Furthermore, it can be challenging to focus the imaging system properly on the DNA strand to ensure all (single-molecule) binding events can be imaged properly for optimal localization.

Improvements to the determination of localization of a binding site of a reagent to the DNA strand are therefore desired.

### SUMMARY

In view of the preceding, herewith is provided a method of detecting a biomolecular process, comprising:
providing a DNA strand comprising one or more luminophores attached to the strand at one or more predetermined positions along the strand;
attaching handles to the DNA strand; and
trapping and/or manipulating the strand by manipulating the handles in a trapping setup.

The method further comprises determining a position of at least one of the one or more luminophores with respect to at least part of the trapping setup;
and determining on the basis of the one or more luminophores one or more of:
a focus position of at least part of an optical detector and/or an optical excitation system;
a focus position of at least part of an optical manipulator;
a position of at least part of the strand;
a position of a portion of the strand relative to one or more other portions of the strand;
a reaction or binding position on the strand of a reagent interacting with the strand.

In the method, the one or more luminophores are covalently attached to the DNA strand.

By covalently attaching the one or more luminophores, risks of detachment and/or relocation of the luminophore along the strand are reduced compared to attaching one or more luminophores via other attachment techniques, such as antibody-antigen labelling (see e.g. Loparo et al., PNAS 108:3584-3589 (2011), DOI: www.pnas.org/cgi/doi/10.1073/pnas.1018824108), enzymatic tagging, etc. Also, covalent binding of a luminophore to the DNA strand may be done well in advance of (an experiment comprising) the attachment of the handles to the strand. The method is therefore beneficial over in-situ labelling of specific sites on a DNA-molecule, e.g. using BsoBI or EcoRV or Cas9 as exemplified in e.g. Heller et al., Chem.Phys.Chem. 15:727-733 (2014), https://doi.org/10.1002/cphc.201300813, since such known methods tend to require extra steps to incubate the DNA molecule with one or more purified proteins and/or guide RNA and such methods may require specific buffer conditions preventing experimental freedom. Also, or alternatively, the binding of such known markers may depend on experimental conditions such as being tension dependent, so that off-target binding may occur and confound the analysis, e.g. see Newton et al. cited above. As a further example it is noted that e.g. EcoRV may bind on Lambda-DNA in 21 different locations, and BsoB1 has 8 binding sites on Lambda DNA; relying on such labelling would therefore lead to a stochastic distribution of labelling sites rather than deterministic positions as presently provided. Moreover, there may be many binding sites for such enzymes which are separated apart less than microscopically resolvable (in particular point spread function) causing inherent uncertainty in any position determination relative to such labelling.

In the presently provided concepts, reliability of position determination on the DNA strand on the basis of the one or more luminophores is improved.

The luminophore may in particular be covalently bound to a single base in a sequence within the DNA strand. Thus, structural properties of the DNA strand may be affected at most to a small extent compared to a DNA strand without the luminophore. As an example, the luminophore may be attached to a thymine within the DNA sequence. Preferably, the luminophore is attached to the DNA sequence so as not to interfere with the double strand structure and/or the helix-structure, e.g. being attached on another side than the interbase-binding side, such as opposite to an interaction side for an interbase-binding. E.g. when the luminophore is attached to thymine, the attachment should preferably be opposite to adenine or an interaction side for interacting with adenine in a double strand DNA.

The luminophore may comprise a fluorescent protein, a quantum dot, a chemiluminescent substance, a bioluminescent substance, a dye, in particular a synthetic fluorophore such as e.g. a dye from the ATTO, Alexa Fluor®, Janelia Fluor® or Cyanine dye families. These synthetic dyes typically provide high brightness (many photons/second) and/or a high photon budget (high average number of photons before bleaching), thus enabling a high localization precision.

The DNA strand may be provided as a basis for studies on the DNA strand itself and/or studies involving interaction of the strand with a reagent. The DNA strand may facilitate and/or simplify studies involving plural reagents. The DNA strand may be at least partly a single-strand DNA strand (ssDNA), but preferably it is at least partly a double-strand DNA (dsDNA).

The predetermined positions may be determined by design and/or manufacture of at least part of the DNA strand, e.g. by attaching the luminophore to a known site in a known sequence, such as by attaching the luminophore to a specific position on a known oligomer that is ligated into the DNA strand. Such attachment may be predetermined to the level of a single base, or single base pair, within a sequence in the DNA strand.

Determining a focus position of the optical detector and/or the optical excitation system on the basis of the one or more luminophores may improve optical studies. In particular, in case of optical studies involving one or more reagents interacting with the strand, both the reagent and the strand may be detected optically and relative positions and/or movements thereof may be determined.

Determining a focus position of the optical detector may comprise focusing the optical detector on the luminophore, e.g. determining a minimum size of the luminophore in an image plane and/or determining a maximum intensity difference in an image plane between the luminophore and an environment thereof. The determination of the focus position may be with respect to one, two or three spatial directions, e.g. lateral and/or along a respective focused beam path. The determination of the focus position of the optical detector may comprise determining a relative position of the one or more luminophores with respect to one or more of the handles.

Also or alternatively, and similar to the preceding, the method may comprise determining a focus position of an illumination system, e.g. for at least partly illuminating the DNA strand. The illumination system may cooperate with, or be part of, the optical detector, e.g. for optical excitation of a luminophore and/or for microscopy techniques such as confocal microscopy, stimulated emission depletion (STED) microscopy, etc.

The optical detector may be configured for singular and/or repetitive detection, e.g. for capturing single images and/or video sequences.

The optical detector and/or the illumination system may also be configured to scan along a one-dimensional scan line in order to allow efficient measurements with high time resolution, for example such as described in U.S. Pat. No. 9,766,180 B2.

The optical detector and/or the illumination system may also, or alternatively, be focussed on a particular spot on the DNA strand. This may facilitate monitoring processes at or near, and/or with respect to, that spot. In combination with a high rate of capturing images, this may facilitate achieving a very high time resolution without a need to scan along the strand.

In case the DNA strand is provided with plural luminophores (see also below), the optical detector and/or illumination system may be focused on a first luminophore while detecting and/or illuminating (possibly exciting) one or more other (second) luminophores. Thus, the first luminophore may be used as a position marker, e.g. for detecting changes in, and/or or associated with, one or more of the second luminophores. Such changes may e.g. be indicative of a movement and/or manipulation of the DNA strand, which may be intentional (e.g. manipulation) and/or provoked (e.g. being a consequence of a method step) and/or accidental.

Similarly, determining a focus position of an optical manipulator may comprise determining a focus position of part of an optical tweezers setup, and/or of part of sample illumination system which may be configured for optical excitation of one or more of a part of the DNA strand, a reagent and a luminophore, etc. This may improve control over the strand and/or the reagent.

In current optical tweezers experiments, focusing is often done on the same signal/molecules as are the molecules under study. This is inefficient because this prevents separation of instrumentation properties and sample properties, e.g. it may prevent determination whether a detected luminescence brightness variation is due to (de) focusing or a behaviour of the molecule(s) of interest. Also or alternatively, data obtained during the focusing may not be optimally usable to study the molecules of interest. Further, the molecules may bleach during the step of bringing the strand in focus. This may reduce or prevent obtaining useful data from such molecules.

Determining a position of at least part of the strand relative to one or more other parts of the strand may comprise determining one or more of an orientation, a length, and a shape of the DNA strand. E.g. one or more of (un-)winding, bending, knotting, and forking of (at least part of) the strand, on purpose and/or by accident, may be determined. In particular, in case the DNA strand is provided with plural luminophores. This may be particularly useful in assays where DNA strands are not tethered along a 1 dimensional straight line but are at least partially bent or knotted e.g. in assays involving more than two optical traps such as shown in, and described with respect to, FIG. 2 of Brouwer et al., Nature, 535 (7613): 566-569 (2016), DOI: https://doi.org/10.1038/nature18643.

In particular, in case the DNA strand comprises plural luminophores, detection of an orientation and/or a length and/or a shape of the strand may be simplified. In such case, a separation of at least some luminophores may be determined which may be used to determine a length of at least part of the strand and/or a length variation of at least part of the strand. At least some of the plural luminophores may have mutually different optical properties, e.g. having different absorption- and/or emission spectra. As an example, this may be used to determine an orientation (direction) of at least part of the strand, e.g. in the sense that it may be determined whether the part of the strand is oriented in one, two or three dimensions. This may be of relevance for an experiment, since in general a sequence of at least part the strand may not be palindromic.

Determining a position on the strand of a reagent interacting with the strand may allow determination of a binding site of the reagent. The reagent may be or comprise proteins and/or protein complexes. To facilitate detection, such proteins and/or complexes may be provided with a fluorophore. E.g. by one or more of labelling with a fluorophore, by labelling with a fluorescent antibody or a quantum dot-modified antibody that recognizes the proteins of interest, by fusing to fluorescent proteins such as GFP.

By determining a position of a reagent relative to a luminophore of the strand, while the luminophore is covalently attached to the strand, a reliable relative position of the reagent and the strand may be determined and thus of the reaction position. E.g. a binding site of a protein to the strand may be determined with improved reliability, improving determination of binding structure and/or binding interactions in general.

The binding position may preferably be determined with a precision of less than 150 base pairs of the DNA strand or better, preferably less than 100 base pairs, e.g. less than 50 base pairs, possibly less than 30 base pairs.

The binding position may preferably be determined with a precision of better than 50 nm, preferably better than 30 nm more preferably better than 15 nm.

Currently, localization accuracy of part of the DNA strand and/or of binding positions of a reagent to the strand is (are) dependent on system calibrations, such as one or more of trap position, handle size (e.g. bead size) and DNA tension, any of which may require calibration in its own right. Such different factors tend to influence the localization accuracy and give it a certain offset and error.

The presently provided method enables determining positions on the basis of the strand itself and thus (at least largely) independent of the system and/or external factors. The one or more luminophores may provide for a calibration measurement under the same conditions, or at least similar conditions, as a measurement on the strand and/or one or more reagents of interest.

In case the strand comprises plural luminophores covalently attached to the strand at predetermined positions in the strand, the luminophores may provide improved spatial determination by providing plural reference positions.

Also or alternatively, the DNA strand may comprise one or more handle moieties for attachment to the handles.

In particular, in case both the handle moieties and one or more sequences of interest elsewhere in the DNA strand comprise luminophores, the luminophore(s) of the handle moieties may differ from the luminophore(s) of the sequence(s) of interest, with respect to one or more optical properties, e.g. having different absorption and/or emission spectra. This facilitates using selective detection. E.g., one or more first luminophores on the handle moieties may be excited by light of one colour, e.g. red light, and one or more second luminophores on or near sequences of interest may be excited by light with another colour, e.g. blue or green light. This may facilitate separate observation of the respective parts during an experiment and excitation of the first or second luminophore(s) may not affect the other (second or first, respectively) luminophore(s). This may e.g. be used to keep the DNA strand in focus of a detection system and/or to deterministically change one or more of the length, tension and position of the DNA strand on the basis of the first luminophores while preserving a limited photon budget of the second luminophores.

The DNA strand may comprise at least one handle moiety of more than 3000 base pairs, in particular more than 4000 base pairs, preferably more than 5000 base pairs such as more than 6000 base pairs. Preferably, the DNA strand comprises handle moieties of substantially equal length on opposite ends. Such long handle moieties facilitate separation of a handle to a sequence of interest in the DNA strand. An end of a handle moiety could be marked with a luminophore and/or other label to simplify distinguishing between the handle moiety and a sequence of interest in the DNA strand.

Manipulating the strand may comprise subjecting at least part of the strand to a predetermined tensile force and/or stretching at least part of the strand.

It has been found that stretching studies on DNA strands may provide valuable information on physical and/or (bio-) chemical properties of the strand. E.g. as indicated above, tension in a strand may affect binding properties of a reagent to the strand, e.g. binding site specificity, binding affinity, binding duration, processivity of a molecular motor, etc. The present method allows improved determination and/or use of such effect. Also or alternatively, tensioning may be used to study (effects of) coiling of the DNA strand and/or assist suppressing thermal fluctuation, e.g. see also WO 2019/139473, WO 2019/098839 and Candelli et al., Phys. Chem. Chem. Phys., 13:7263-7272 (2011).

By having the one or more luminophores attached covalently, e.g. ligated and incorporated by covalent bonds, the one or more luminophores are integrated into the DNA strand structure itself, at least to a high degree. Therefore, the attachment is robust against manipulation and stretching and the luminophore(s) will stay attached and not move along the strand. Also, the DNA strand may have a structural integrity comparable to a DNA strand without the one or more luminophores. The position of a luminophore along the DNA strand thus may be well known and stable under the manipulation so that it is a reliable beacon and which may serve as a reference for determination of other positions.

The luminophore may be attached to the DNA strand via a linker that is covalently attached to the DNA strand. E.g. an ATTO647N fluorophore may be covalently attached to thymine using a linking moiety (or commonly: “linker”), e.g. having a length of 5-25 atoms, preferably in a range 10-20 atoms such as in a range 10-15 atoms. Note that, the shorter the linker, the better position determination of the DNA strand with respect to the luminophore may be. Suitable ATTO647N-labeled nucleotides with short linkers are available commercially.

A plurality of luminophores may allow determining of a distance between them. Therefore, accurate relative position information may be obtained and/or information regarding DNA length and/or stretching, thus also binding characteristics of a reagent related to DNA-length, stress, matching sizes, etc. E.g. plural luminophores in a single strand may serve as a ruler of sorts along the DNA strand, possibly allowing detection of uneven stretching of one or more parts of the strand relative to other parts of it.

Any embodiment may comprise determining a reaction position on the strand of a reagent interacting with the strand, wherein one or more luminophores are attached to the reagent, preferably covalently attached, and/or wherein the interaction of the reagent with the strand is associated with an optical process allowed to affect or caused to affect at least one luminescent property of at least one of the luminophores attached to the DNA strand.

When the reagent also comprises a luminophore and/or is associated with an optical process affecting a luminophore of the DNA strand, the reagent's position may be determined with high reliably relative to one or more of the luminophores on the DNA strand. The reaction position of a reagent interacting with the strand may be determined before, during and/or after a reaction, e.g. binding of a protein to the DNA strand.

This may allow restricting the field-of-view of an imaging system to a precise location on the strand where one or more bindings and/or are expected, which may enable more efficient data collection and/or data collection at a higher time resolution. For example, the region-of-interest of an sCMOS or EMCCD camera may be limited only to a known location on the strand of just one or a few pixels wide, which enables acquisition at high frame rates. Similarly, a scanning laser excitation beam (e.g. a confocal excitation beam, a STED excitation beam or a multiphoton excitation beam) may be restricted to scan over only a very small region, e.g. scanning over a distance of less than a micron, and/or such laser beam may even be parked on a single location for only illuminating the location of interest, this may facilitate very high time resolutions.

An optical process affecting a luminophore of the DNA strand may comprise affecting at least part of an absorption spectrum and/or of an emission spectrum (e.g. changing an emission colour), affecting a bleaching behaviour (e.g. accelerating bleaching), affecting a blinking behaviour, and/or allowing or rather inhibiting luminescence of the luminophore of the DNA strand altogether (switching, quenching, energy transfer, etc.). Such interaction may signal a binding behaviour of the reagent to the DNA strand.

Attachment of handles to the DNA strand may comprise attaching distinguishable handles to the strand, preferably optically distinguishable handles. Suitable handles may be microbeads for optical tweezers, magnetic/magnetizable objects, electrically chargeable objects etc.

Also, or alternatively, at least some of the luminophores may have mutually distinguishably different optical properties, e.g. having different absorption and/or emission spectra, scattering.

This allows or simplifies determining a direction of at least part of the DNA strand on the basis of the respective different optical properties. Thus, this allows or simplifies detecting and/or arranging a particular direction of the DNA strand and/or of a sequence within the strand. The method may comprise providing handles of different materials and/or handles comprising different functional groups. In particular in such case, the DNA strand may be provided with handle moieties being different for selective attachment to the different materials and/or the different functional groups. E.g. one end of the DNA strand may be provided with biotin, whereas an opposite end of the DNA strand may be provided with digoxigenin, for selective attachment of streptavidin and, respectively, anti-digoxigenin provided beads to the respective ends as different handles. Distinguishability of the handles may e.g. be due to one or more of size, shape, colour, reflectivity, transmissibility or opaqueness, magnetic response, etc. Different handles may also be deterministically picked up by different traps of a multi-trap setup such as an optical tweezers setup. E.g. the respective beads may be selected using a laminar flow system as shown in, and described with respect to, FIG. 7B of WO 2014/196854 A1, so that for example one trap contains a streptavidin-coated bead while the other trap contains an anti-digoxigenin-coated bead, and the DNA strand is trapped with a well-determined directionality.

Any embodiment may comprise determining a time dependent behaviour of at least one of
a position of at least part of the strand;
a position of a portion of the strand relative to one or more other portions of the strand; and
a reaction position on the strand of a reagent interacting with the strand.

Thus, better understanding of the respective behaviour and/or the respective behaviours with respect to each other may be obtained.

The determination of the time dependent behaviour may comprise repetitive determination of the respective position associated with determining time periods.

Determining a time dependent behaviour may be facilitated by visualising results of repeated determinations in a kymograph and/or a movie clip. Also or alternatively, determining a time dependent behaviour may comprise determining binding durations and/or determining dwell time. Analysis of determined data may comprise recording and/or analysis of moving and/or static images and use of statistical techniques such as histograms.

In any embodiment the step of providing the DNA strand may comprise one or more luminophores covalently attached to the strand at one or more predetermined positions along the strand may comprise
the steps of
providing an oligomer;
covalently attaching a luminophore to the oligomer thus providing an optically labelled oligomer;
providing a double strand DNA strand;
replacing a sequence from the DNA strand with the optically labelled oligomer.

This may facilitate determining a location of the luminophore on and/or in the DNA, in particular relative to at least another part of the DNA strand. Attaching a luminophore to an oligomer facilitates determining the location of the luminophore, e.g. since oligonucleotides can be easily synthesized and labelled with a fluorophore in a specific position of the oligonucleotide sequence.

Further, replacing a sequence in a double strand DNA by an oligomer, in particular for non-palindromic sequences and oligomers, provides a well-determined position of the oligomer into the DNA strand, e.g. by requiring multiple base pair matchings between the oligomer and an associated part of the DNA strand, and therefore a position of the luminophore may be determined with a high precision. E.g. Lambda DNA can be labelled using a short fluorophore-modified oligonucleotide which is complementary with a unique sequence in the Lambda DNA that can be removed from the Lambda dsDNA after nicking. The fluorophore-modified oligonucleotide can be incorporated instead of the removed sequence into the Lambda DNA. Subsequent ligase covalently binds the oligonucleotide to the Lambda DNA. Thus, the fluorophore is ultimately covalently attached the DNA sequence of Lambda.

The DNA strand may comprise a lesion at a predetermined position with respect to the position of the one or more luminophores. Thus, the lesion and any effect thereof on an interaction with a reagent may be determined accurately, whereas the lesion itself may not be visible or otherwise detectable during an experiment. Note that such DNA strand may comprise a plurality of lesions each at a predetermined position with respect to the position(s) of the one or more luminophores.

It is noted that also or alternatively, DNA strands may be compared that have each identical sequences and have each a luminophore covalently attached to the same base, but otherwise differing in that a first one of the DNA strands to be compared has a second luminophore covalently attached to it at a second location along its strand, whereas another one of the DNA strands to be compared has a lesion at the second location along its strand, instead of the second luminophore. Thus, the location of the (invisible) lesion can be more accurately determined by comparison.

In view of the preceding, herewith also a DNA strand is provided for any method disclosed herein.

The DNA strand comprises a plurality of luminophores covalently attached to the strand at a plurality of predetermined positions along the strand, and/or the DNA strand comprises one or more luminophores covalently attached to the strand and one or more lesions at a predetermined position with respect to the position of the one or more luminophores. The DNA strand is provided with handle moieties for attachment to trappable and/or manipulatable handles, e.g. polystyrene beads for optical trapping, magnetic beads for magnetic trapping, etc.

By providing the plurality of luminophores and/or the combination of one or more luminophores and one or more lesions, as specified, accurate position determination of at least part of the DNA strand and/or a reagent is facilitated. By provision of the handle moieties, the DNA strand may be provided with the handles for the trapping and/or manipulating.

The handle moieties may be different for selective attachment to distinguishable handles. This allows or simplifies detecting and/or arranging a particular direction of the DNA strand and/or of a sequence within the strand, as indicated before.

The plurality of luminophores may be attached substantially equidistant along the DNA strand.

Also or alternatively, the DNA strand may comprise a plurality of substantially the same sequences, each of the sequences being provided with one or more of the plurality of luminophores covalently attached to the sequence; then, the plurality of substantially the same sequences may be oriented the same along the DNA strand. In another embodiment the DNA strand may comprise a plurality of different sequences, each of the sequences being provided with one or more of the plurality of luminophores covalently attached to the sequence.

The sequences may be assembled using methods known in the art. The assembly may comprise several steps or may be done in a single step. The sequences may be ligated to the handle moieties using methods known in the art. In case, the DNA strand comprises a plurality of sequences, the luminophore(s) attached to each sequence may be the same of may be different. The luminophore(s) may be attached to the C-terminal end, the N-terminal end or anywhere else within a sequence.

Either or both simplifies detection of (changes in) separation between at least some of the luminophores. This also facilitates studying properties and/or behaviour of the strand under manipulation of the strand and/or attachment of a binding moiety to the strand. In particular, this may allow studying differences in properties and/or behaviour of different portions of the strand when subject to tension.

The equidistance may be determined in terms of numbers of base pairs and/or sequences and/or functional units along the strand, e.g. genes.

At least some of the luminophores may have mutually different optical properties, e.g. having different absorption and/or emission spectra, scattering. Also or alternatively, the strand may be provided with handles having mutually different optical properties, e.g. having different absorption and/or emission spectra, scattering.

Either or both may assist identifying directionality of the DNA strand and/or identifying particular (groups of) luminophores, which may assist identifying particular portions and/or sequences of the DNA strand and/or simplify determining one or more of position, shape and length of at least part of the strand.

### BRIEF DESCRIPTION OF THE DRAWINGS

The above-described aspects will hereafter be more explained with further details and benefits with reference to the drawings showing a number of embodiments by way of example.

FIGS. 1, 1A and 2 schematically indicate method steps of providing a DNA strand as disclosed herein.

FIG. 3 schematically indicates an experimental system for performing part of the method as disclosed herein.

FIGS. 4A, 5A and 6A are confocal microscopy images of DNA strands as disclosed herein provided with handles and being optically trapped and tethered.

FIGS. 4B, 5B and 6B are schematic depictions of the DNA strand imaged in FIGS. 4A, 5A and 6A, respectively. FIGS. 5C, 5D indicate method steps of providing such DNA strand.

FIG. 7A shows a two-colour confocal fluorescence 2D image of a DNA molecule tethered between two optically trapped beads. FIG. 7B is a negative grayscale version of FIG. 7A.

FIG. 7C is an intensity plot of two colour channels of the confocal fluorescence image of FIGS. 7A, 7B.

FIG. 7D is a kymograph of the setup of FIGS. 7A-7C.

FIG. 8A-8D indicate different methods of determining a Cas9-binding site from kymographs as in FIG. 7D.

FIG. 9 indicates determining a position of a luminophore of the DNA strand from the position of other luminophores of the DNA strand.

FIG. 10 is kymograph showing dynamic binding processes.

FIG. 11A schematically describes an example of constituents of the kit of parts as described herein. The constituents include two handle moieties each provided with biotin molecules for attachment to streptavidin-conjugated handles and each provided with a luminophore, and a control DNA strand. FIG. 11B describes a sequence of interest (SOI) ligated to the two handle moieties shown in FIG. 11A allowing tethering of the SOI between two handles trapped in, for example, an optical tweezer system.

FIG. 12 schematically shows the pUC-LUMICKS plasmid. The pUC-LUMICKS plasmid is a high-copy plasmid containing the pMB1 origin for replication in E. coli. The multicloning site (MCS) is in frame with the LacZalpha gene, allowing white-blue screening for insertion of the sequence of interest (SOI) by alpha complementation. The map shows the enzymes with unique restriction sites in the MCS, which can be used for the insertion of the SOI. The TypeIIS enzymes BsaI, BbsI, and BsmBI can be used to cut the SOI and generate overhangs compatible for ligation with handle moieties.

### DETAILED DESCRIPTION OF EMBODIMENTS

It is noted that the drawings are schematic, not necessarily to scale and that details that are not required for understanding the present invention may have been omitted. The terms “upward”, “downward”, “below”, “above”, and the like relate to the embodiments as oriented in the drawings, unless otherwise specified. Further, elements that are at least substantially identical or that perform an at least substantially identical function are denoted by the same numeral, where helpful individualised with alphabetic suffixes.

Further, unless otherwise specified, terms like “detachable” and “removably connected” are intended to mean that respective parts may be disconnected essentially without damage or destruction of either part, e.g. excluding structures in which the parts are integral (e.g. welded or molded as one piece), but including structures in which parts are attached by or as mated connectors, fasteners, releasable self-fastening features, etc.

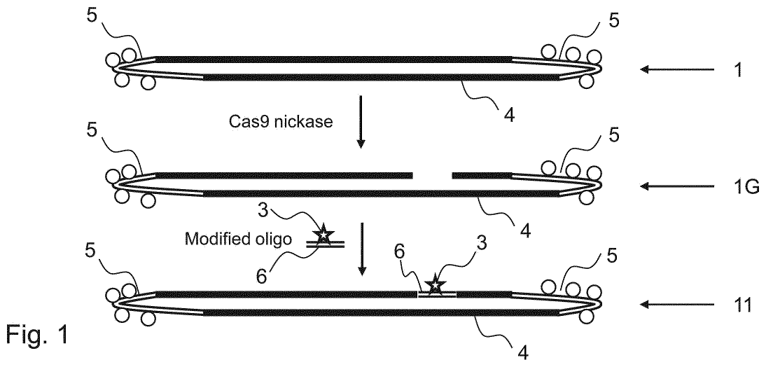

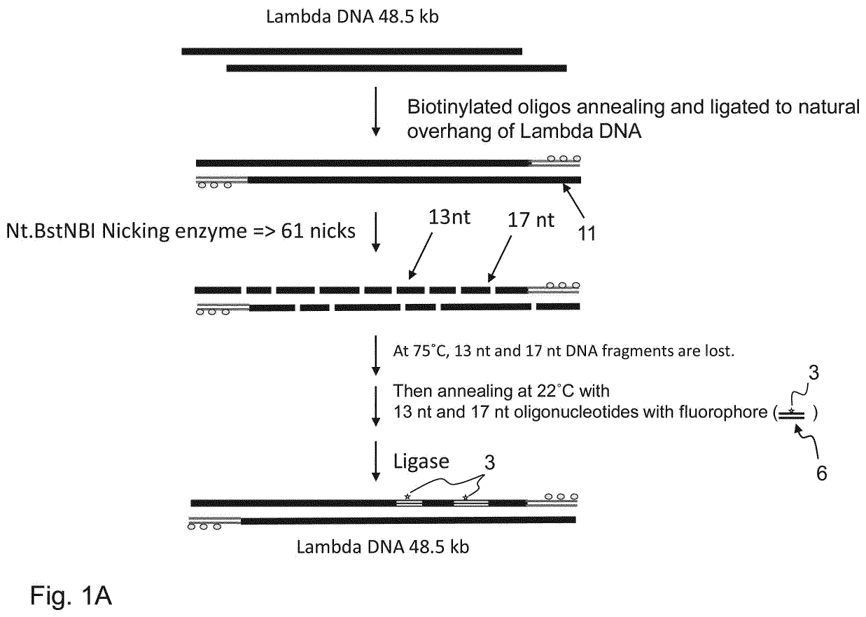

FIG. 1 schematically indicates method steps of providing a DNA strand 1 with a luminophore 3 at a predetermined position in the strand 1 to provide a DNA strand 11 comprising the luminophore 3 attached to the strand 11 at a predetermined position along the strand.

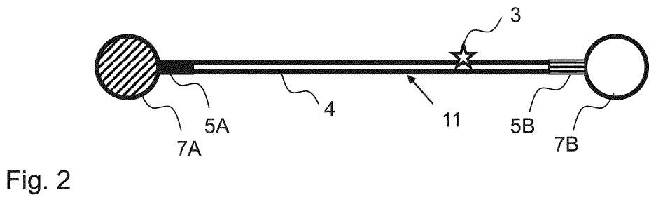

In step 1, indicated in the top row, a double strand DNA strand (“dsDNA”) molecule 1 is provided. For simplicity, a helical structure of the dsDNA is not indicated. The dsDNA strand 1 comprises a main section 4 which is provided with handle moieties 5 on opposite ends of the section 4, e.g. one or more biotins on each end. FIG. 2 schematically indicates a DNA strand provided with different handle moieties such as a biotin-containing sequence 5A on one end and a digoxigenin-containing sequence 5B on another end, to which handles 7A and 7B are attached, see also below.

In step 2, the dsDNA strand 1 is nicked. E.g., using Cas9 nickase, which is highly position specific (second row). Also or alternatively, other nicking endonucleases could be used, e.g, Nt.BstNBI. The nicking defines a short single stranded fragment in one of the single strands of the dsDNA strands (not shown). The ss-strand is liberated from the dsDNA providing a gapped dsDNA 1g (third row).

Next, modified oligomers 6 are provided (fourth row), which oligomers have the same sequence as the liberated ss-strand. The modified oligomers are provided with a luminophore 3 covalently attached to a base in the oligomer sequence. Typically, oligomers with a length of 10 nt to 30 nt may be used, preferably with a length 13 nt to 24 nt.

The modified oligomers are annealed into (the gaps of) the gapped dsDNA and ligased into the dsDNA strand. Thus, a luminophore-containing dsDNA strand 11 is provided, comprising a luminophore covalently attached to the strand at a predetermined position along the strand (fifth row). Here, the luminophore is located offset from a middle of the strand, at about 30% of the length of the dsDNA strand.

In another example, shown in FIG. 1A, an initial dsDNA strand is provided, e.g. a Lambda DNA of 48.5 kb length. As an option, the initial dsDNA strand is first provided with biotinylated oligomers by annealing and ligating to the natural overhangs of the Lambda DNA (top row).

Next (second row), the dsDNA strand was nicked in plural positions using Nt.BstNBI. By incubating at 75° C. for 2 minutes DNA fragments of 13 nt and 17 nt are lost, providing the DNA strand with plural gaps. Then, plural oligomers each complementary to the remaining single strand sequence of the DNA strand and comprising a fluorophore such as ATTO647N (commercially available), were annealed by slowly cooling down from 75° C. to 22° C. at a ramp rate of 0.2° C. per second into the thus-formed gaps so that the resultant modified dsDNA strand comprised plural luminophores covalently attached to the strand at predetermined positions along the strand.

Note that any luminophore-containing oligomer ligated into the DNA strand as described above may itself serve as, or provide, a sequence of interest and/or it may serve to provide the luminophore into the DNA strand 11 for reference to a/another sequence of interest.

The dsDNA may be plasmid dsDNA or lambda phage DNA, and the handles could be provided to naturally occurring ssDNA overhang ends, e.g. using dNTPs.

Next, after preparation of the luminophore-containing DNA strand 11, handles 7 are attached to the luminophore-containing DNA strand 11, as indicated in FIG. 2. In FIG. 2, each individual handle 7A, 7B is a bead provided with conjugate moieties to the respective different handle moieties 5A, 5B of the strand 11. The beads 7A, 7B may be the same or may be distinguishable, e.g. being at least partially of different material or a different size (see different hatchings).

The beads 7A, 7B may be trapped in optical traps provided by focused laser beams thus tethering the DNA strand 10 between the beads. The trapping system may, e.g., be a system as generally discussed in WO 2014/196854, WO 2016/129994 or in WO 2020/130812.

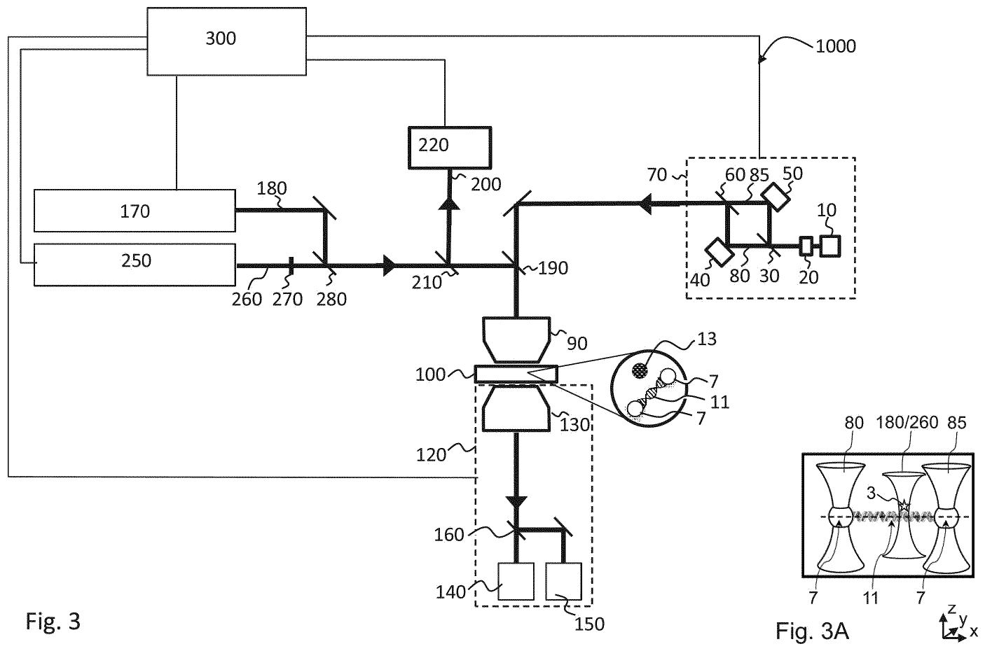

E.g. FIG. 3 schematically illustrates an embodiment of a suitable trapping and microscopy system 1000.

The system 1000 comprises a sample holder 100 for holding a sample, a trap system 70, as options an excitation system 170 and a depletion system 250, an imaging detector 220 and a controller 300 as an option being connected to each of these systems (only partly shown).

The optical system 1000 comprises a trap system 70 for establishing an optical trap that can hold an object such as a bead 7 to which an object such as a DNA strand 11 may be connected; in particular, the trap system may be configured for a dual optical trap holding two objects 7, such as beads, simultaneously with the DNA strand 11 connected between them, as shown in the enlarged detail in FIG. 3 and in FIG. 3A.

Optical traps are known in the prior art. An example of an optical trap is described in Ashkin et al., Opt Lett 11:288 (1986). doi: 10.1364/OL.11.000288, and in the afore-mentioned WO 2014/196854, WO 2016/129994 and WO 2020/130812. Typical bead sizes ranges used in optical experiments may vary from 100 nanometer to 5 micrometer.

In one embodiment, the trap system 70 comprises a trap light source 10 for generating trapping light. The trap light source 10 may be a laser, e.g. a 1064 nm CW laser, although numerous other types of laser sources are suitable as well. Further, trap system 70 may comprise various optical elements such as a module 20 for rotating a polarization of the trapping light and a polarizing beam splitter 30 for splitting the trapping light into two light beams 80, 85, one for establishing a first trap and a second for establishing a second trap. The light beams are transmitted through an optical system (not indicated) comprising optical elements such as lenses, mirrors, polarizers, pinholes, etc. to direct and shape the respective beam 80, 85 suitably for forming a trap. In each trap, the trapping light 80, 85 provides an optical trapping force onto the bead 7 by which the bead 7 is held in the trap; see FIG. 3A.

In addition, the trap system 70 may comprise a module 40 for controlling the position of the first trap and a module 50 for controlling the position of the second trap. In particular, independent trap steering may be done via a coarse-positioning piezo stepper mirror in one of the modules 40, 50 and an accurate piezo mirror for the respective traps, in the other one of the modules 40, 50. A (polarizing) beam splitter 60 may be used to recombine the individually controlled trapping beams. The trapping beams are focused by an objective 90 into the sample holder 100 containing a sample, here containing beads 7 with the strand 11. If the trap system 70 is configured to establish two traps, as shown, advantageously the object of interest in the sample, here: the DNA strand 11, can be held fixed between two optically trapped object such as beads 7. The position of each bead may be determined and/or controlled in one, two or, preferably, three dimensions X, Y, Z as indicated in FIG. 3A. As discussed in more detail in WO 2014/196854, in an embodiment, one or more telescopes (not shown) may be present in the optical path for properly projecting the beams from (the tip/tilt mirrors of) the modules 40 and 50 onto the back focal plane of the objective 90. Thus, the respective beam may be expanded and/or convergence of the beam may be controlled. This may facilitate overlaying a trapping focal plane and an imaging focal plane onto each other (see also below).

As another option, not shown, the trap system may be configured for one or more other optical traps, e.g. a quadruple optical trap for holding four objects 7, such as beads, simultaneously as discussed in WO 2016/129994, wherein the DNA strand 11 and one or more sample portions may be connected between respective ones of the objects. In the system, one or more, preferably each of the traps may be manipulated independently. Such arrangement also allows engagement and/or entangling and/or knotting of two DNA strands each connected to two beads. Note that entangling and/or knotting may comprise three-dimensional manipulation (X, Y, Z-direction); the present methods and systems facilitate detecting, monitoring and/or controlling position and/or movement of parts of the respective DNA strands in all three dimensions due to the luminophore(s). Covalent attachment of a luminophore, instead of other attachment techniques, such as antibody-antigen labelling, reduces risks of detachment from the DNA strand during manipulation for such entanglement and/or knotting.

The shown optical system 100 comprises, as an option, a force detection system 120, that is configured to detect a force exerted by at least one of the traps established by trap system 70 on the trapped object. The force detection system 120 shown comprises a condenser lens 130 that collimates the trapping beams and directs them towards a force detection module 140 for detecting a force exerted by the first trap and optionally a force detection module 150 for detecting a force exerted by the second trap. As known in the art, these modules 140 and 150 may be position dependent sensors as the force can be determined based on a deflection of the trapping light and using back-focal plane interferometry. A polarizing beam splitter 160 may be used to separate the trapping beams from the first and second traps.

Thus, the system allows subjecting at least part of the strand 11 to a predetermined tensile force and/or stretching at least part of the strand 11. However, it is noted that according to the present concepts, a tensile force need not be known while one or more positions and/or separations of (parts of) the DNA strand and/or objects relative to the DNA strand may still be reliably determined due to the luminophore(s).

The optical system 1000 may further comprise an illumination optical system. In particular, as shown an excitation optical system comprising an excitation light source 170 may be provided, such as an excitation laser having a suitable wavelength for exciting a luminophore of the DNA strand. In particular, for exciting dye molecules of ATTO647N, a laser having a wavelength of 639 nm may be used. The excitation optical system 170 may be configured to focus excitation light 180 onto the sample portion of interest (here: strand 11) through the objective, for example by reflection of an optional dichroic mirror 190. The excitation light 180 may excite molecules and/or one or more luminophores 3 in the sample, possibly dependent on a tight focusing or not of the excitation light beam 180; see FIG. 3A. A focus position of the excitation light 180 relative to at least part of the DNA strand 11 may be determined by determining the focus position relative to at least one luminophore 3 of the DNA stand 11, e.g. in a direction X along the DNA strand, and/or in a direction Z along a direction of propagation of the beam of excitation light 180 and/or in a direction Y perpendicular to both the strand and the direction of propagation of the beam of excitation light 180 (see bold arrows in FIG. 3A). In case of a laser scanning imaging system, such as e.g. a confocal laser scanning system, one or more beam scanning optical elements such as e.g. scan mirrors may be provided including any required relay telescopes or lens systems. The excited molecules may subsequently decay and emit fluorescence light. Fluorescence emission 200 may be collected by the objective 90 and may be directed towards a detector 220, e.g. by dichroic mirrors 190 and 210. The detector may comprise an avalanche photodiode, a photomultiplier, but a (possibly digital) camera or other detector is also conceivable.

As an option, the optical system 1000 may be configured for STED microscopy. E.g., the system 1000 may then further comprise a depletion light source 250, e.g. a laser, in particular, for depleting excited molecules ATTO647N a laser having a wavelength of 767 nm may be used. The depletion light source 250 may generate a depletion beam 260 which passes through an optional optical beam shaping element, e.g. a phase mask 270, which may imprint a desired intensity pattern on the beam, e.g. determining a local intensity minimum in the beam focus. In the shown example, the beam path of the depletion beam 260 is combined and overlapped with the beam path of the excitation beam 180 at a dichroic mirror 280, but a polarizing beam splitter and/or another combiner may also be used. This may simplify arranging an overlap of a focus of the depletion beam 260 and of a focus of the beam of excitation light 180 (FIG. 3A). As discussed in more detail in WO 2014/196854 one or more telescopes (not shown) may be present in the optical path of the illumination and/or detection system in order to properly project the beams from any beam scanning mirrors onto the objective back focal plane, to possibly expand the beam and to control the convergence of the beam and thereby allow the imaging focal plane to be overlayed onto the trapping focal plane.

Also, one or more further optical systems for excitation and/or detection may be provided.

The sample may comprise reagents 13, e.g. proteins which may be provided with luminophores.

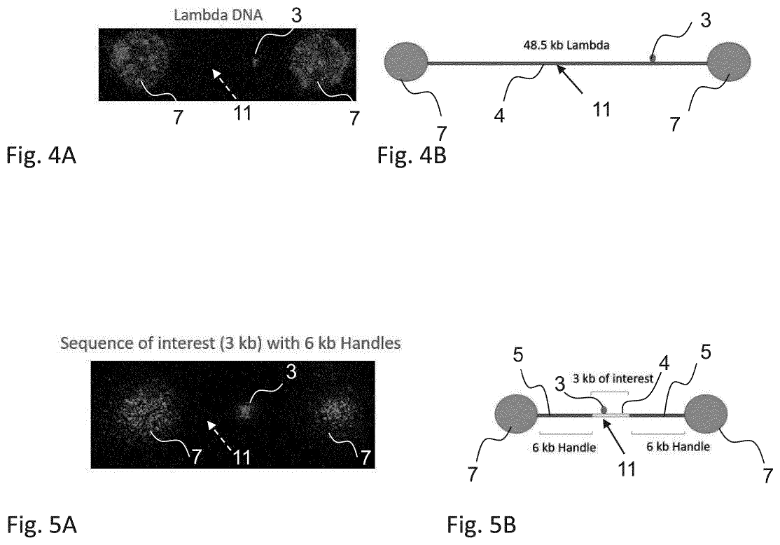

The system allows optical detection of the luminophore of a DNA strand and possibly also optical detection of the beads between which the DNA is tethered; e.g. FIGS. 4A and 4B show a microscopy image and a schematic depiction, respectively, of a 48.5 kb Lambda phage DNA comprising a single luminophore, as an example: ATTO647N. In FIG. 4A, the sample was illuminated with red (laser) light at 658 nm, causing the fluorescence captured in the image. The position of the luminophore is clearly discernible, whereas the shapes and positions of the beads 7 are less well defined. The DNA strand itself is not visible.

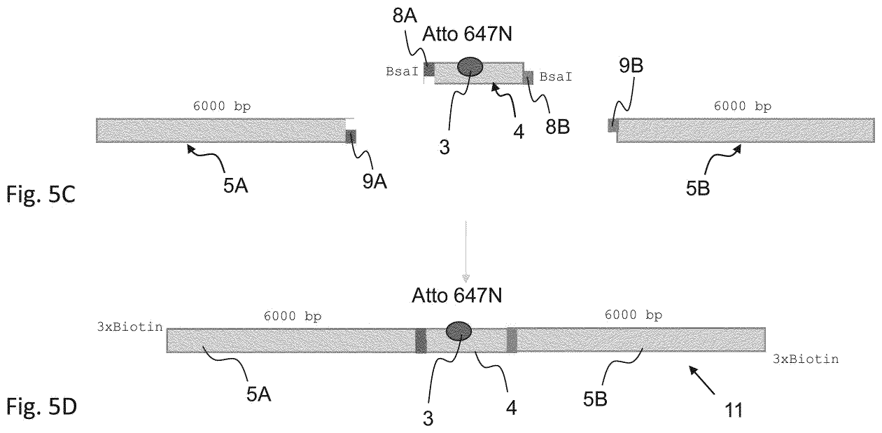

FIGS. 5A, 5B show, respectively, like FIGS. 4A, 4B a (fluorescence) microscopy image and a schematic depiction of a DNA strand 11 comprising a single luminophore 3 (again: ATTO647N illuminated with 658 nm light as example, but it will be apparent to the reader that other luminophores and/or excitation light colours may be used suitably). Here, the DNA strand 11 comprises a main section 4 that is a relatively short sequence of only 3 kb length; however, the handle moieties 5A, 5B are relatively long, about 6 kb. The length of the handle moieties allows separation between the handles (beads 7) and the sequence of interest; thus increasing detectability of the luminophore and preventing interaction between the handles and the sequence of interest. Note that the handle moieties may be configured to prevent interaction between a handle sequence and a reagent in the sample (not shown). FIGS. 5C, 5D indicate method steps of providing such DNA strand.

In FIG. 5A, is visible that the luminophore fluoresces in red light whereas the beads serving as handles for optical tweezers autofluoresce in green light under the excitation illumination. Such spectral difference increases contrast between the handles and the luminophore, further improving detection of the luminophore and/or any reactions affecting luminescence of the luminophore.

FIG. 5C shows a dsDNA section 4 provided with a covalently bound luminophore 3 and having overhanging ssDNA ends 8A, 8B, which may be palindromic but preferably are non-palindromic. Here, the luminophore 3 is ATTO 647N, but other luminophores may be used. Further, handle moieties 5A, 5B are indicated, each comprising having overhanging ssDNA ends 9A, 9B, complementary to the ends 8A, 8B.

FIG. 5D shows that the handle moieties 5A, 5B are ligated to the dsDNA section 4 by connection of the respective overhanging ends 8A, 9A; 8B, 9B, thus forming a dsDNA strand 11 comprising a luminophore 3.

The handle moieties 5A, 5B are long, here they are 6000 base pairs long, as indicated. Here, each of the handle moieties 5A, 5B is provided with 3 biotin molecules, for attachment to streptavidin-conjugated handles (see FIGS. 5A, 5B).

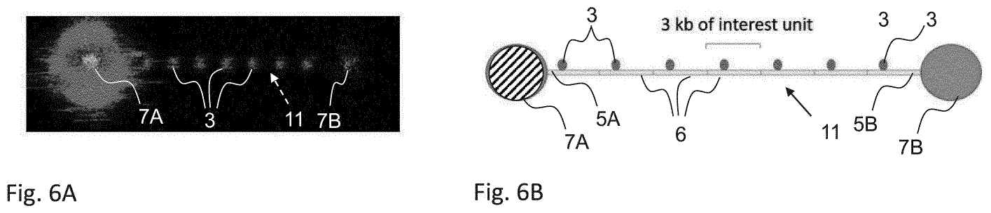

FIGS. 6A, 6B show respectively, a microscopy image and a schematic depiction of a DNA strand 11 comprising a plurality of luminophores 3. The DNA strand comprises a tandem repeat of oligomers of about 3 kb length. Further, the strand 11 is provided with different handle moieties 5A, 5B on opposite ends, e.g. digoxigenin and biotin, respectively. Optically distinguishable handles 7A, 7B have each been provided with different attachment moieties for attachment to the handle moieties, e.g. anti-doxigenin and streptavidin, respectively, and these have been attached to the strand 11. Thus, when trapped, the directionality of the DNA strand is detectable, see FIG. 6A. In this example, the luminophores appear to be arranged equidistant along the strand 11, indicating that the oligomers are unidirectionally arranged in the DNA strand 11.

A tethered DNA strand may be analysed in various ways as known in the art, e.g. by making kymographs and/or intensity line scans of a series of successive images. The one or more luminophores may provide a reference in at least part of the images, e.g. facilitating alignment of successive images and/or line scans thereof. Thus facilitates reduction or removal of experimental imprecision and/or noise, e.g. by improved averaging. Displacement of a protein with respect to a luminophore may be identified reliably.

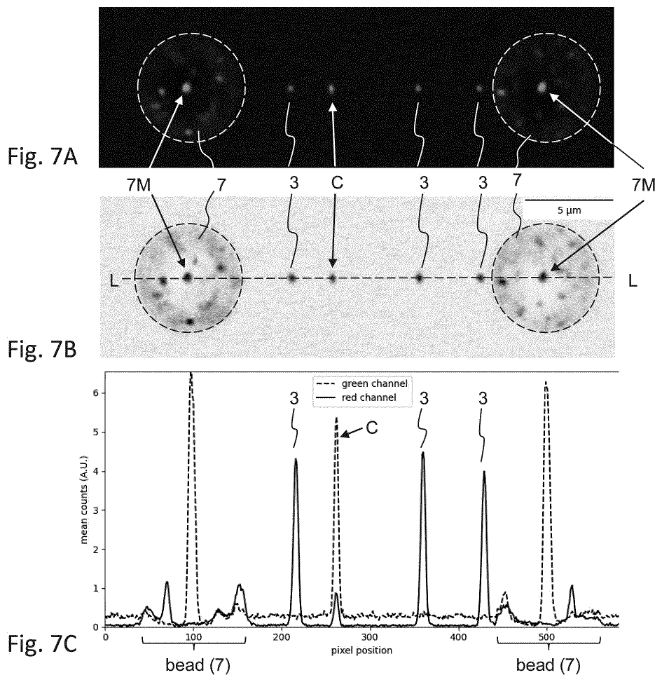

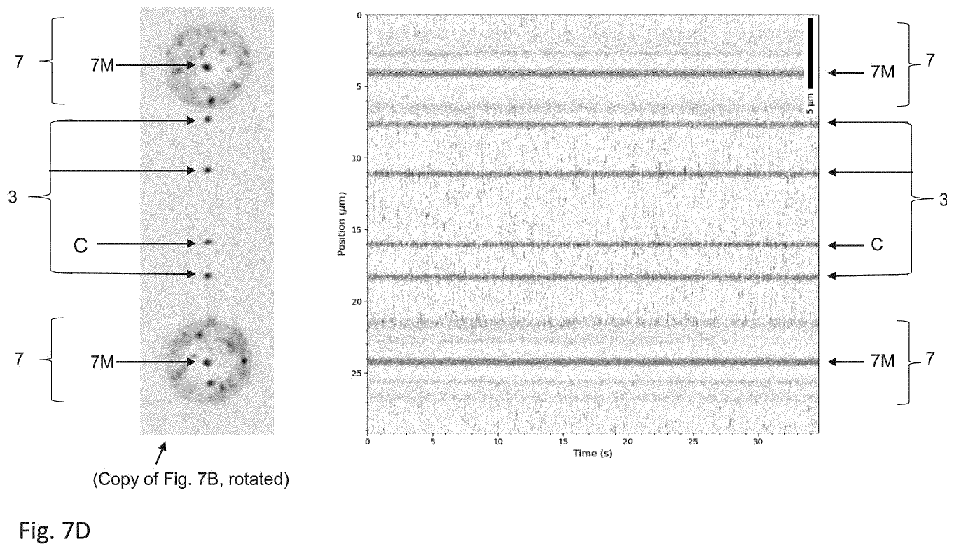

For instance FIG. 7A shows a 2D image (like FIGS. 4A, 5A, 6A) of a DNA molecule tethered between two optically trapped beads 7 (bead diameter 4.38 micrometer). The DNA molecule is a Lambda dsDNA strand and includes three fluorescent markers as luminophores 3 (red, ATTO647N). The beads 7 show autofluorescence at their respective centres 7M. A Cas9 molecule labelled with another fluorophore (green, ATTO565) is bound to its target site along the DNA strand; the location is indicated with “C”. In a colour image the difference is clearly visible. FIG. 7B is a grayscale negative image of FIG. 7A; also here the respective luminophores 3 and (the fluorescent label of) the Cas9 are clearly visible. FIG. 7C indicates optical intensity of red and green light, respectively (red: solid line; green: dashed line) along a trace L in the image of FIG. 7A/7B, typically a line scan along the line of highest intensity. Such analysis may be done on the basis of camera images and/or on the basis of images of dedicated detectors which may comprise line scanners. FIG. 7C shows the result of separate detectors used to analyse the red and green light respectively (“red channel”; “green channel”). Since in the used setup part of the fluorescence of the ATTO565 is detectable in the red channel as well due to its emission spectrum, the position of the Cas9 is visible in both detector signals (here, at about pixel position 260). This may serve for (checking) alignment of both traces.

FIG. 7D shows a grayscale kymograph over more than 30 seconds of the DNA strand with the Cas9 molecule attached to it. For ease of reference, the image of FIG. 7B is included as well, rotated by 90 degrees. Clearly, the relative positions of the luminophores are constant, reflecting a stable configuration of the DNA strand and the Cas9 bound to the DNA strand. From the line traces (FIG. 7C) and/or from the kymograph (FIG. 7D) positions of the respective portions may be determined, e.g. as follows:

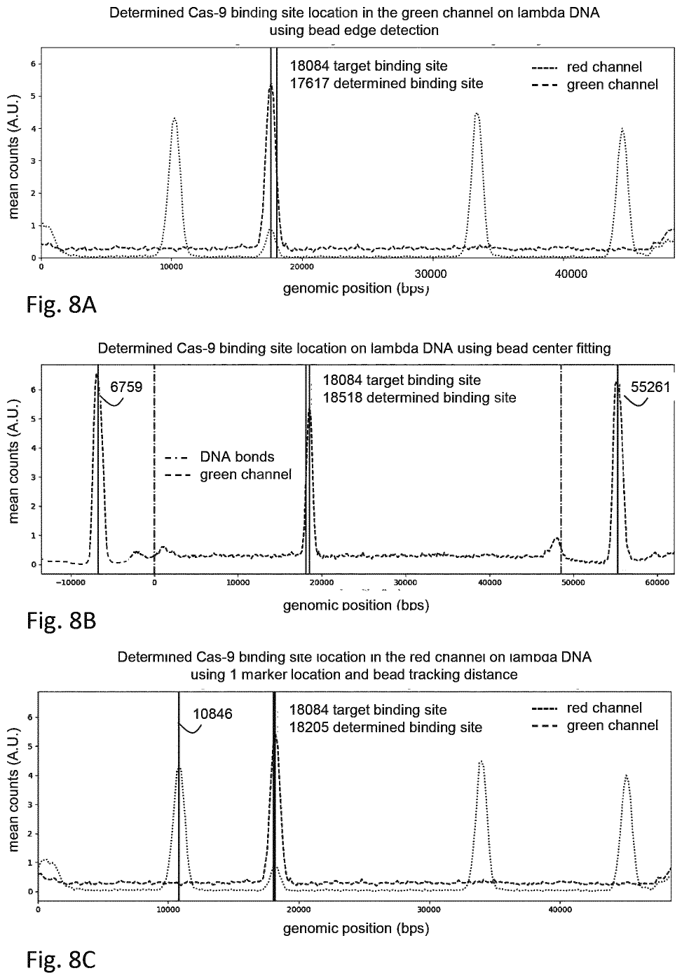

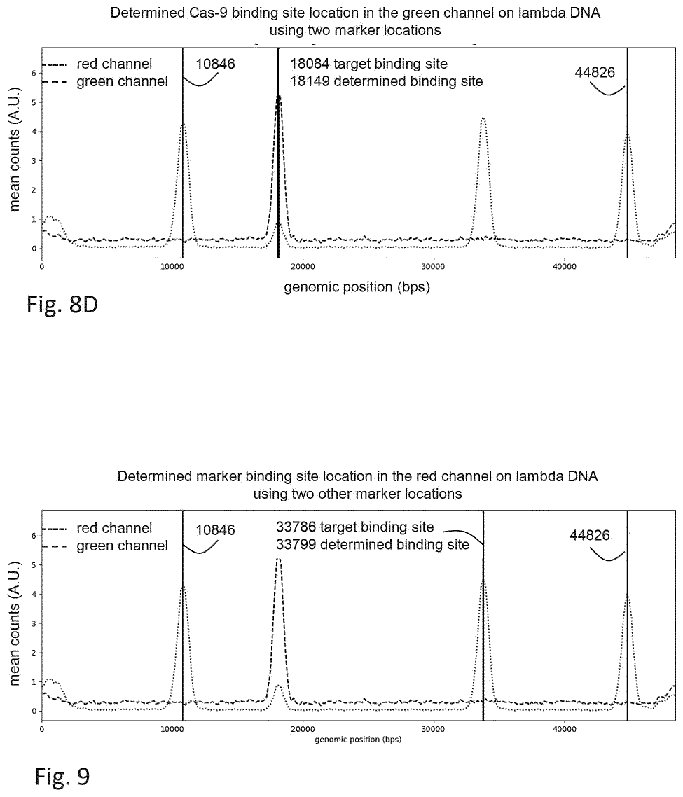

Each of FIGS. 8A-8D and 9 indicates an intensity profile as in FIG. 7C from kymographs as shown in FIG. 7D. In the experiments, the used lambda DNA strand and the Cas9 molecule were the same. The thee ATTO647N fluorophore molecules were covalently attached to Thymine bases in the DNA strand at genomic positions 10846, 33786 and 44826, respectively. The target binding site of the Cas9 on the lambda DNA was predetermined to be at genomic position 18084 along the strand. Further, in the used the experimental conditions and employing about 10 pN tensile force to the DNA strand 11, a length scale of 0.34 nanometers per base pair of the DNA strand applies.

In FIG. 8A the binding site of the Cas9 was determined using edge detection of the autofluorescence of the beads 7. The autofluorescence of the beads 7 is unpredictable and could lead to higher inaccuracies when the autofluorescence signal is relatively low. The determined Cas9 binding site using this method was at genomic position 17617. This method therefore leads to a determined binding site with an offset of 467 base pairs (or: about 159 nm) from the target binding site.

In FIG. 8B, the binding site of the Cas9 was determined based on fitting the intensity peaks on the bead centers 7M. The intensity peaks on the beads are not always visible which makes this an unreliable method to use for localization determination. The determined Cas9 binding site using this method was at genomic position 18518. This method therefore leads to a determined binding site with an offset of 434 base pairs (or: about 148 nm) from the target binding site.

In FIG. 8C, the binding site of the Cas9 was determined based on fitting the intensity peak from 1 fluorescent marker (i.e. a luminophore 3) and using the distance between the beads 7 from bead tracking in the brightfield camera image (not shown). Determining the distance between the beads from the bead tracking is dependent on brightfield versus confocal distance calibration. The determined Cas9 binding site using this method was at genomic position 18205. This method therefore leads to a determined binding site with an offset of 121 base pairs (or: about 41 nm) from the target binding site.

In FIG. 8D, the binding site of the Cas9 was determined based on fitting the intensity peak from two fluorescent markers (i.e. two luminophores 3). The determined Cas9 binding site using this method was at genomic position 18149. This method therefore leads to a determined binding site with an offset of 65 base pairs (or: about 22 nm) from the target binding site.

Clearly, use of one luminophore as provided herein significantly increases accuracy of determining a binding site compared to known methods. Use of two luminophores as provided herein provides yet another significant improvement over using only one luminophore.

In FIG. 9, as a further check of the present concepts, the location of one of the luminophores was determined based on the positions of two other luminophores; in particular the position of the middle marker was determined using the intensity profiles of the two outer fluorescent markers to show the accuracy of the localization determination method. The luminophore under consideration was known to be at genomic position 33786. The determined position using this method was at genomic position 33799. This method therefore leads to a localization determination of the fluorescent marker with an offset of only 13 base pairs (or: about 4 nm) from the actual position.

Without wishing to be bound to any particular theory, it is believed that the further accuracy of the determination of the middle marker (FIG. 9; 13 base pairs offset) compared to the accuracy of the Cas9 binding site (FIG. 8D; 65 base pairs offset) in the present test-experiments may be the result of experimental origins such as noise and/or alignment inaccuracies between the red- and green-wavelength detectors in the used setup, and/or the result of inherent origins such as structural artefacts of the respective Cas9 molecule and its fluorophore and/or an offset in the actual DNA strand binding site of the fluorescent moiety of the Cas9, which may be at least partly resolved in further research and experimental and/or by appropriate correction when processing the experimental data (e.g. using a correction factor and/or software development).

The position of a lesion on the DNA may strand replacing a luminophore may therefore also be determined with very high precision.

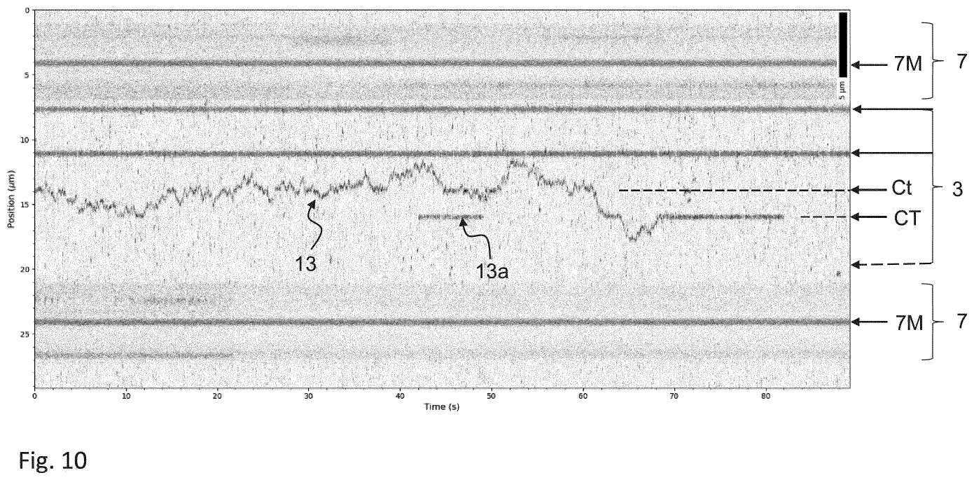

FIG. 10 indicates use of the method for determining and/or quantifying binding kinetics. FIG. 10 is a kymograph of spanning about 80 seconds of a same DNA molecule provided with covalently attached luminophores 3 (red, ATTO647N; two luminophores 3 active, one bleached but its known position indicated with a dashed line) tethered between two beads 7 (4.38 μm diameter) as in FIGS. 7A-9. In FIG. 10 is also visible (a trace of) an optically labelled (green, ATTO565) Cas9 molecule 13 which is movably bound to the DNA strand, but not tightly bound to its target site. Clearly visible in the Figure, during the experiment the Cas9-molecule 13 moves along the DNA strand, “searching” until it locates its target site and binds to that at and after about 69 seconds of the kymograph; the target site is marked CT.

Also is visible that at a time of about 41 seconds, another Cas9 molecule (also optically labelled in the same way—indicated as 13a) binds to the Cas9-target site of the DNA strand, producing two Cas9 traces (green in a colour image, see separate indication). However, that other Cas9 molecule 13a detaches again from the DNA strand and disappears from view at a time of about 50 seconds. Then, the target binding side is left free for subsequent occupation by the Cas9 molecule 13 considered from the start in the kymograph.

Further, it may be discerned that at the position marked Ct the Cas9 molecule 13 under consideration appears to have a larger dwell time than elsewhere along the DNA (except for the target binding site). This may indicate a less-preferred binding site; modification of experimental conditions, e.g. stretching or relaxing the DNA strand somewhat may transfer such less-preferred binding site to a strongly-preferred binding site or the other way around (cf. the paper by Newton (2019) cited in the beginning).

From FIG. 10 may be understood that the luminophores 3 covalently attached to the DNA strand provide a constant and reliable reference for determination interaction location, binding location and/or dynamics of molecules and/or other reagents with the DNA strand. A further advantage of using multiple covalently attached luminophores as references is that this provides some redundancy in that when some of the luminophores bleach or are otherwise absent others may still be used as reference marker.

Also provided herein is a kit of parts. In an embodiment the kit of parts as described herein can be used in the methods of detecting a biomolecular process as described herein and/or can be used for making a DNA strand (11) as described herein.

The constituents of the kits of parts as described herein may be stored in one or more containers prior to their application and/or use. In an embodiment the kit of parts is stored at a temperature below 0° C., e.g. −20° C. Containers include tubes, such as Eppendorf tubes, or vials. The kits of parts as described herein may be multi-pack kits, wherein different components are stored in a plurality of containers. In an embodiment each of the components of the kit of parts are stored in a separate container. In an alternative embodiment a container may comprise two or even more components of the kit of parts.

In an embodiment the components of the kit of parts as described herein are present in the containers at a millilitre (ml) or microliter (μl) volume, for example at a volume of 1 μl-50 μl. In case the kit of parts comprises several containers, the volume of each container may be identical, but may also differ.

A kit of parts according to the invention comprises at least two handle moieties. The handle moieties comprise a DNA sequence. In an embodiment the at least two handle moieties are labelled, e.g. biotinylated. In an embodiment each of the at least two handle moieties is a biotin-containing DNA sequence. In an embodiment each of the at least two handle moieties has at least one biotin molecule on one end of the DNA sequence, preferably at the 3-end of the sequence. In an embodiment at least one of the at least two handle moieties has two or more biotin molecules on one end of the DNA sequence, preferably at the 3-end of the sequence. In an embodiment all handle moieties have two or more biotin molecules on one end of the DNA sequence, preferably at the 3-end of the sequence. One or more handle moieties may have two, three four, five, six or even more biotin molecules on one end of the DNA sequence, preferably at the 3-end of the sequence. Examples of handle moieties are handle moieties 5A and 5B as shown in FIG. 2, handle moieties 5 as shown in FIG. 5B and handle moieties 5A and 5B as shown in FIGS. 5C and 5D.

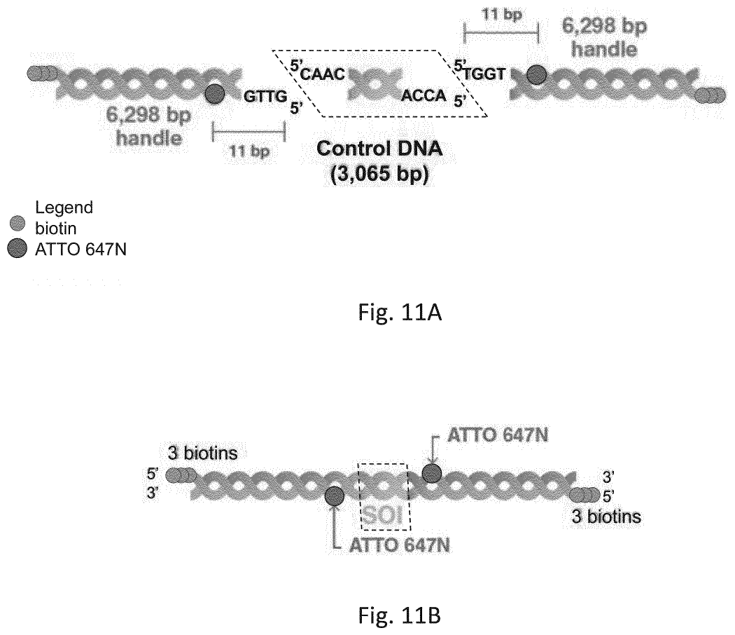

In an embodiment each of the at least two handle moieties comprises an overhanging ssDNA end at one end of the DNA sequence. In an embodiment the overhanging ssDNA end is a 3′-overhang. In another embodiment the overhanging ssDNA end is a 5′-overhang. In an embodiment the overhanging ssDNA end is complementary to an overhanging ssDNA end of a control DNA sequence. In an embodiment the at least two handle moieties differ in that they comprise a different overhanging ssDNA end. Examples of the overhanging ssDNA ends are ssDNA sequences 9A and 9B as shown in FIG. 5C. ssDNA sequences 9A and 9B are complementary to the ends 8A and 8B on a control DNA. Further examples of suitable overhanging ssDNA ends on the control DNA sequence and the handle moieties can be found in FIG. 11A.

In an embodiment the at least two handle moieties have a length of more than 100 base pairs, preferably more than 500 base pairs, even more preferably more than 1000 base pairs, yet even more preferably more than 3000 base pairs, in particular more than 4000 base pairs, even more in particular more than 5000 base pairs such as more than 6000 base pairs. In an embodiment the at least two handle moieties have a length of 100-20,000 base pairs, in particular 3000-10,000 base pairs, more in particular 4000-9000 base pairs, even more in particular 5000-8000 base pairs such as 6000-7000 base pairs. In an embodiment the at least two handle moieties are of equal length, i.e. have the same number of base pairs. In another embodiment the at least two handle moieties have a different length.

As described above, each of the handle moieties may comprise a biotin molecules which can be used for attachment of the handle moieties to streptavidin-conjugated handles. In an embodiment the handles are distinguishable handles, preferably optically distinguishable handles. Preferably, the handles are trappable and/or manipulatable handles. Suitable handles may be microbeads for optical tweezers, magnetic/magnetizable objects, electrically chargeable objects etc. Examples of suitable include, but are not limited to, polystyrene beads for optical trapping, magnetic beads for magnetic trapping, etc.

In an preferred embodiment the at least two handle moieties are marked with one or more luminophores and/or other labels to simplify distinguishing between the handle moiety and a sequence of interest (SOI) or a control sequence. In an embodiment the one or more luminophores and/or other labels are located at a predetermined position of each of the two handle moieties. The position may be the same on each of the two handle moieties, but the position may also differ between the two handle moieties.

In an embodiment the one or more luminophores and/or other labels are covalently attached to the handle moieties. The luminophore may be attached to the handle moieties via a linker that is covalently attached to the handle moiety. In an embodiment the at least two handle moieties are marked with the same luminophore and/or other label. In an alternative embodiment the at least two handle moieties are marked with a different luminophore and/or other label. In an embodiment the luminophore marking each of the at least two handle moieties is an ATTO647N fluorophore. This fluorophore may be covalently attached to thymine using a linking moiety (or commonly “linker”), e.g. having a length of 5-25 atoms, preferably in a range 10-20 atoms such as in a range 10-15 atoms. In another embodiment one of the at least two handle moieties is marked with an ATTO647N fluorophore, while the other of the at least two handle moieties is marked with an ATTO488 fluorophore.

The luminophore may be covalently bound to a single base in the sequence within the handle moieties. The luminophore may comprise a fluorescent protein, a quantum dot, a chemiluminescent substance, a bioluminescent substance, a dye, in particular, a synthetic fluorophore such as e.g. a dye from the ATTO, Alexa Fluor®, Janelia Fluor® or Cyanine dye families. In an preferred embodiment the at least two handle moieties are marked with a dye of the ATTO family.

In an embodiment the at least two handle moieties are present in a separate container. In another embodiment the at least two handle moieties are present in the same container.

The kit of parts as described herein may also comprise a control DNA sequence. The DNA sequence may be at least partly a single-strand DNA sequence (ssDNA), but preferably it is at least partly a double-strand DNA sequence (dsDNA). In an embodiment the control DNA sequence is present in a container at a concentration of 1-100 ng/μl, preferably 25-75 ng/μl.

In an embodiment the control DNA sequence has a length of more than 20 base pairs, preferably more than 100 base pairs, even more preferably more than 500 base pairs, in particular more than 1000 base pairs, more in particular more than 2000 base pairs and even more in particular more than 3000 base pairs. In an embodiment the control DNA sequence has a length of 20-20,000 base pairs, preferably 100-15,000 base pairs, more preferably 500-10,000 base pairs, in particular 1000-6,000 base pairs, more in particular 2000-5000 base pairs, even more in particular 3000-4000 base pairs. In an embodiment the control DNA sequence is marked with a luminophore and/or other label, but preferably the control DNA sequence is not marked with a luminophore and/or other label.

In an embodiment the control DNA sequence comprises an overhanging ssDNA end at both ends of the sequence. In an embodiment each of the overhanging ssDNA ends is a 3′-overhang. In an embodiment each of the overhanging ssDNA ends is a 5′-overhang. In an embodiment the sequence of each of the overhanging ssDNA ends of the control DNA sequence differs. In an embodiment the overhanging ssDNA ends of the control DNA sequence are complementary to the overhanging ssDNA ends of the handle moieties. Examples of the overhanging ssDNA ends of the control DNA sequence are ssDNA sequences 8A and 8B as shown in FIG. 5C. ssDNA sequences 8A and 8B are complementary to the ends 9A and 9B on handle moieties. Further examples of suitable overhanging ssDNA ends on the control DNA sequence and the handle moieties can be found in FIG. 11A. As described before, the handle moieties and the control DNA sequence provided with the kit contain complementary overhangs. Ligation of the control DNA sequence to the handle moieties is highly efficient and can be used as a positive control of the DNA ligase reaction. To ligate the sequence of interest (SOI) to the handle moieties, the SOI requires the same overhangs of the control DNA sequence. These overhangs allow efficient ligation without by-products due to the self-ligation of either DNA handle moieties or SOI and without the ligation of one handle moiety to the other handle moieties.

In an embodiment the kit of parts as described herein may further comprise a component selected from the group consisting of DNA ligase, ligase buffer, handles, plasmid and any combination thereof.

In an embodiment the ligase buffer is concentrated ligase buffer such as 10× ligase buffer.

In an embodiment the handles are streptavidin-coated polystyrene beads. In an embodiment the beads have a diameter of from 0.2-10 μm, preferably of from 0.5-5 μm, in particular of from 1-2.5 μm.

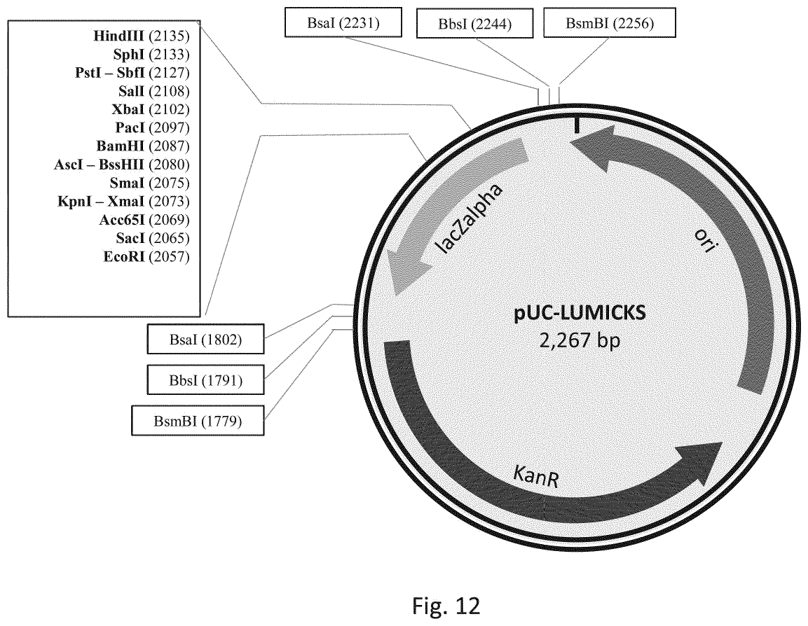

In an embodiment the plasmid is a pUC-LUMICKS plasmid as shown in FIG. 12.

A kit of parts as described herein may further comprise instructions for using the one or more components of the kit of part. For example, the kit of parts may comprise instructions for the DNA ligase reaction; instructions for purification of the SOI; instructions for buffer exchange; instructions for making overhangs that are compatible for the ligation to the handle moieties by means of PCR or using a plasmid; instructions for experimental set-up for the microfluidics system with a laminar flow cell; instructions for catching beads; instructions for DNA tethering; and instructions for measuring enzymatic activity.

In an embodiment the kit of parts as described herein comprises:
a container comprising two handle moieties, each handle moiety comprising a luminophore, at least one biotin molecule at one end of the handle moiety and a single stranded DNA overhang on the other end of the handle moiety, wherein the single stranded DNA overhang differs between the two handle moieties,
a container comprising a control DNA sequence, said sequence comprising a single stranded DNA overhang at both ends, wherein the single stranded DNA overhang differs between the two ends,
optionally, a container comprising streptavidin-coated handles,
optionally, a container comprising a DNA ligase enzyme,
optionally, a container comprising a DNA ligase buffer, and
optionally, a container comprising a plasmid.

In an embodiment the kit of parts as described herein comprises:
a container comprising two handle moieties, each handle moiety comprising a luminophore, at least one biotin molecule at one end of the handle moiety and a single stranded DNA overhang on the other end of the handle moiety, wherein the single stranded DNA overhang differs between the two handle moieties,
a container comprising a control DNA sequence, said sequence comprising a single stranded DNA overhang at both ends, wherein the single stranded DNA overhang differs between the two ends,
a container comprising streptavidin-coated handles,
a container comprising a DNA ligase enzyme,
a container comprising a DNA ligase buffer, and
a container comprising a plasmid.

The kit of parts as described herein is a highly efficient system to tether a DNA sequence of interest (SOI) in, for example, an optical tweezer system and study dynamics of, for example, DNA-binding proteins. In an embodiment the sequence of interest may comprise a plurality of sequences. The sequences may be the same of may differ. In an embodiment some or all of the sequences are provided with one or more luminophores. In an embodiment the luminophores are covalently attached to the sequence. The sequences may be assembled using methods known in the art. The assembly may comprise several steps or may be done in a single step. The sequences may be ligated to the handle moieties using methods known in the art. In case, the sequence of interest comprises a plurality of sequences, the luminophore(s) attached to the individual sequences may be the same of may be different. The luminophore(s) may be attached to the C-terminal end, the N-terminal end or anywhere else within the individual sequences.

Using the kit of parts as described herein, the SOI can be ligated to two handle moieties (see FIG. 11B). In FIG. 11B each handle moiety is 6.3 kbp long (exact size 6,298 bp) and can be linked to the sequence to be inserted. Each handle moiety is labelled with an ATTO fluorophore, a biotin molecule on one end and a 4 nucleotides single stranded DNA overhang on the other end. To allow ligation of the SOI to the handle moieties, overhangs complementary to the overhangs of the handle moieties have been introduced in the SOI. The overhangs can be introduced in the SOI by digestion with a TypeIIS restriction enzyme, for example BsmBI, BbsI or BsaI. These enzymes cleave outside of their recognition sequence allowing to choose the sequence of the overhangs formed upon digestion. TypeIIS restriction sites can be introduce in the SOI either by PCR or by using the pUC-LUMICKS plasmid included in the kit.

Once the SOI is ligated to the two DNA handles, the biotin moieties of each handle moiety will allow tethering of the SOI between two streptavidin-coated beads trapped in optical tweezers. In this configuration, the ATTO fluorophores will flank the SOI (at a defined position from the ligation sites) and can be used to position the SOI on the focal plane before incubation with fluorescent proteins. This enables setting up optimal fluorescence imaging conditions before starting a DNA-protein interaction measurement, which allows capturing even the first interactions. More importantly, as the distance between the two fluorophores is known, this can be used as a ruler on the tethered DNA to precisely determine the position on the DNA sequence of fluorescent proteins interacting with the SOI.

In addition to the handle moieties, a kit of parts as described includes a DNA ligase enzyme, 10×DNA ligase buffer, a 3 kbp control DNA with overhangs (exact size 3,065 bp), and a plasmid (pUC-LUMICKS link) to insert the SOI between TypeIIS restriction sites. A kit of parts may provide material for more than one DNA ligase reactions of the SOI to the DNA handle moieties. Each DNA ligase reaction can be used for at least 5 experimental sessions on the optical tweezer system called C-Trap® (from Lumicks).

The disclosure is not restricted to the above-described embodiments which can be varied in a number of ways within the scope of the claims. Elements and aspects discussed for or in relation with a particular embodiment may be suitably combined with elements and aspects of other embodiments, unless explicitly stated otherwise.

1. A method of detecting a biomolecular process, comprising
providing a DNA strand comprising one or more luminophores attached to the DNA strand at one or more predetermined positions along the DNA strand;
attaching handles to the DNA strand;
trapping and/or manipulating the DNA strand by manipulating the handles in a trapping setup;
wherein the method further comprises:
determining a relative position of at least one of the one or more luminophores and at least part of the trapping setup; and
determining on the basis of the one or more luminophores one or more of:
a focus position of at least part of an optical detector and/or an illumination system;
a focus position of at least part of an optical manipulator;
a position of at least part of the DNA strand;
a position of a portion of the DNA strand relative to one or more other portions of the DNA strand; and
a reaction or binding position on the DNA strand of a reagent interacting with the DNA strand; and
wherein the one or more luminophores are covalently attached to the DNA strand.

2. The method according to claim 1, wherein one of the one or more luminophores is covalently attached to a single base in the DNA strand.

3. The method according to claim 1, wherein the manipulating the DNA strand comprises subjecting at least part of the DNA strand to a predetermined tensile force and/or stretching at least part of the DNA strand.

4. The method according to claim 1, comprising determining the reaction position on the DNA strand of the reagent interacting with the DNA strand,
wherein the one or more luminophores are attached to the reagent, and/or
the interaction of the reagent with the DNA strand is associated with an optical process allowed to affect or caused to affect at least one luminescent property of at least one of the luminophores attached to the DNA strand.

5. The method according to claim 1, wherein attaching the handles comprises attaching distinguishable ones of said handles to the DNA strand.

6. The method according to claim 1, wherein at least some of the luminophores have mutually different optical properties, scattering, and/or the DNA strand is provided with the handles and the handles have mutually different optical properties, and determining a direction of at least part of the DNA strand on the basis of the respective different ones of said optical properties.

7. The method according to claim 1, comprising determining a time dependent behaviour of at least one of:
the position of at least part of the DNA strand;
the position of a portion of the DNA strand relative to the one or more other portions of the DNA strand; and
the reaction position on the DNA strand of the reagent-interacting with the DNA strand.

8. The method according to claim 1, wherein the step of providing the DNA strand comprising the one or more luminophores covalently attached to the DNA strand at the one or more predetermined positions along the DNA strand comprises steps of:
providing an oligomer;
covalently attaching the luminophore to the oligomer thus providing an optically labelled oligomer;
providing a double stranded DNA strand; and
replacing a sequence from the double stranded DNA strand with the optically labelled oligomer.

9. The method according to claim 1, wherein the DNA strand comprises a lesion at a predetermined position with respect to the position of the one or more luminophores.

10. A DNA strand for the method of claim 1, comprising a plurality of luminophores covalently attached to the DNA strand at a plurality of predetermined positions along the DNA strand, and/or comprising one or more of the luminophores covalently attached to the DNA strand and one or more lesions at respective positions predetermined with respect to the position of the one or more luminophores, and wherein the DNA strand is provided with handle moieties for attachment to trappable and/or manipulatable handles.

11. The DNA strand according to claim 10, wherein the handle moieties are different for selective attachment to distinguishable ones of the handles.

12. The DNA strand according to claim 10, wherein the one or more luminophores are attached substantially equidistant along the DNA strand.

13. The DNA strand according to claim 10, wherein the DNA strand comprises a plurality of substantially identical sequences, each of the sequences being provided with one or more of the luminophores covalently attached to the sequence, and the plurality of substantially identical sequences are oriented the same along the DNA strand.

14. The DNA strand according to claim 10, wherein at least some of the one or more luminophores have mutually different optical properties, and/or the DNA strand is provided with handles having mutually different optical properties.

15. A kit of parts comprising:
a container having two handle moieties, each handle moiety including a luminophore, at least one biotin molecule at one end of one of the handle moiety and a single stranded DNA overhang on another end of the handle moiety, wherein the single stranded DNA overhang differs between the two handle moieties,
a container having a control DNA sequence, said control DNA sequence including a single stranded DNA overhang at both ends, wherein one of the single stranded DNA overhangs differs from the other single-stranded DNA overhang.

16. The method according to claim 4, wherein the one or more luminophores are covalently attached to the reagent.

17. The method of claim 5, wherein the handles are optically distinguishable handles.

18. The method of claim 6, wherein the optical properties are selected from the group consisting of absorption and/or emission spectra and scattering.

19. the method of claim 10, wherein the trappable handles are polystyrene beads for optical trapping and the manipulatable handles are magnetic beads for magnetic trapping.

20. The method of claim 14, wherein the optical properties are selected from the group consisting of absorption and/or emission spectra and scattering.
